(19) United States
(12) Patent Application Publication
(10) Pub. No.: US 2007/0299350 A1
(43) Pub. Date: Dec. 27, 2007

(54) Blood pressure monitor with removable power source

(76) Inventors: Mordechai Lev, West Bloomfield, MI (US); Roman S. Ferber, West Bloomfield, MI (US); Robert T. McCulloch, Macomb, MI (US)

Correspondence Address: BROOKS KUSHMAN P.C., 1000 TOWN CENTER, TWENTY-SECOND FLOOR, SOUTHFIELD, MI, 48075, US

(21) Appl. No.: 11/448,483

(22) Filed: Jun. 7, 2006

(51) Int. Cl. A61B 5/02

(52) U.S. Cl. 600/485

(57) ABSTRACT

A blood pressure monitor is disclosed with a housing and controls provided within the housing that are in communication with an external blood pressure sensor. A display is oriented on the housing for receiving the blood pressure measurement and displaying it for external viewing. A removable power source is selectively attachable to the housing for powering the controller and the display.

### BACKGROUND OF THE INVENTION

1. Field of the Invention

This invention relates to blood pressure monitors.

2. Background Art

Individuals having health problems relating to blood pressure are extremely common, and thus routine monitoring of blood pressure is necessary. A blood pressure measurement is taken by temporarily stopping the flow of blood in an artery, typically by inflating a cuff around the upper arm, and listening for the sound of the blood beginning to flow through the artery again as air is released from the cuff. Sounds continue to be heard until pressure in the artery exceeds the pressure in the cuff. Blood pressure is thus recorded as two measurements: 1) systolic pressure, which is the pressure of the blood flow when the heart beats (the pressure when the first sound is heard); and 2) diastolic pressure, which is the pressure between heartbeats (the pressure when the last sound is heard). Blood pressure measurements are recorded as systolic pressure/diastolic pressure.

An individual does not need to have his or her blood pressure measured at a doctor's office, but can rather take a blood pressure measurement at home using a consumer blood pressure monitor. Home monitoring of blood pressure is especially useful when an individual requires blood pressure measurements to be taken frequently. In addition to monitoring blood pressure at home, people may desire to take their blood pressure monitors along with them on vacation or even carry the blood pressure monitor with them in daily travels such that they can constantly have the ability to check their blood pressure measurement if necessary. However, consumer blood pressure monitors may be bulky and/or heavy, thus making their frequent transport undesirable.

### BRIEF DESCRIPTION OF THE DRAWINGS

FIG. 1 is a front perspective view of a blood pressure monitor according to the present invention;

FIG. 2 is a rear perspective view of a blood pressure monitor according to the present invention with a removable power source mounted theron;

FIG. 3 is a rear perspective view of a blood pressure monitor according to the present invention with a removable power source disconnected;

FIG. 4 is a schematic view of a blood pressure monitor according to the present invention;

FIG. 5 is a rear perspective view of a blood pressure monitor according to the present invention with a removable power source mounted thereon; and

FIG. 6 is a rear perspective view of a blood pressure monitor according to the present invention with a removable power source disconnected.

### DESCRIPTION OF THE INVENTION

As required, detailed embodiments of the present invention are disclosed herein; however, it is to be understood that the disclosed embodiments are merely exemplary of the invention that may be embodied in various and alternative forms. The figures are not necessarily to scale, and some features may be exaggerated or minimized to show details of particular components. Therefore, specific structural and functional details disclosed herein are not to be interpreted as limiting, but merely as a representative basis for the claims and/or as a representative basis for teaching one skilled in the art to variously employ the present invention.

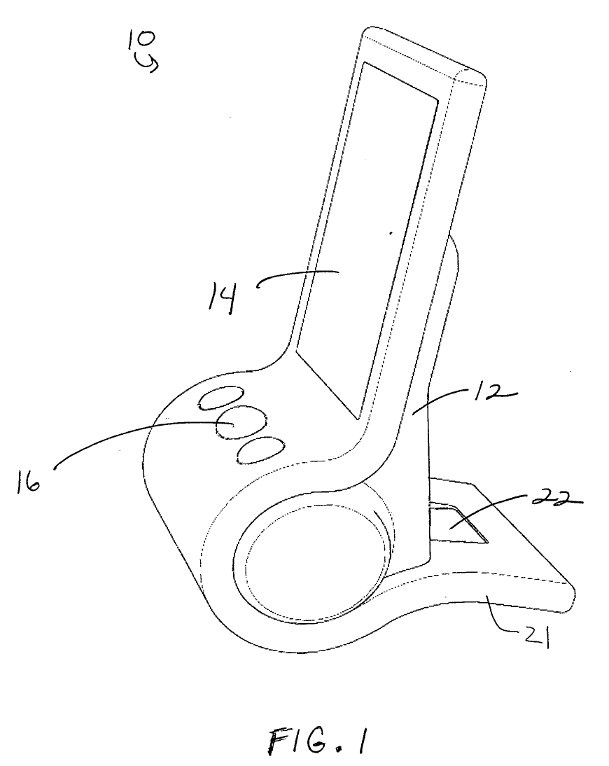

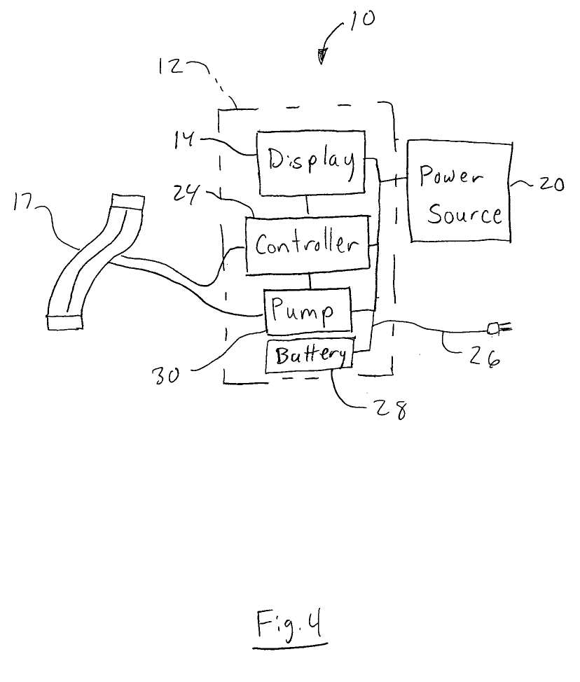

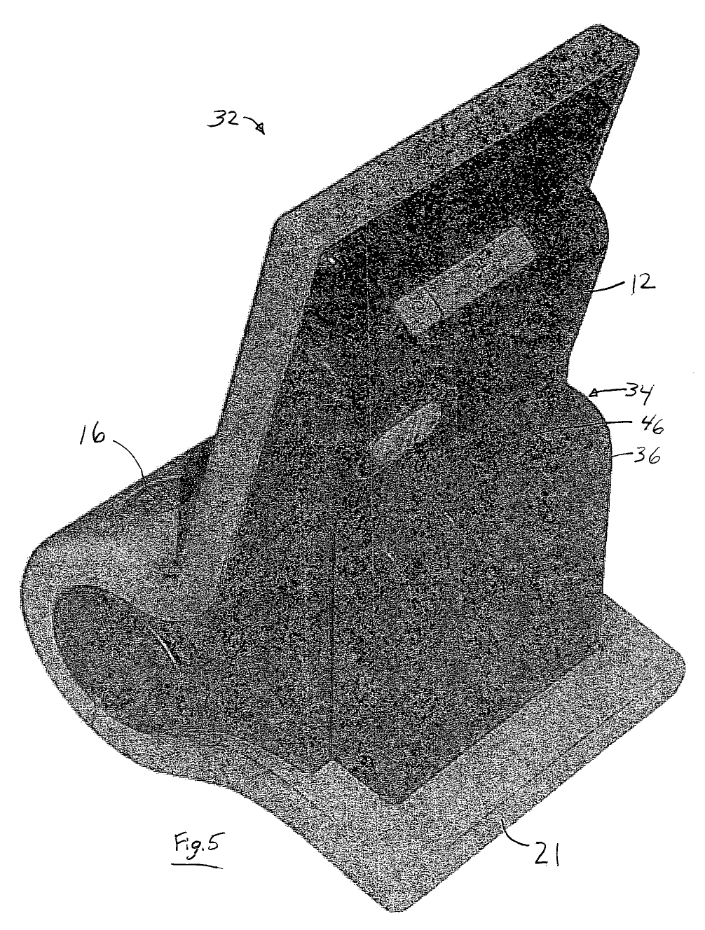

The present invention includes a blood pressure monitor having a removable power source such as, but not limited to, a battery which may provide increased versatility of the blood pressure monitor for a user. With reference first to FIGS. 1 and 4, a blood pressure monitor 10 according to the present invention may include a housing 12 having a display 14 and various control buttons 16, and may operate in accordance with any consumer blood pressure monitor. Blood pressure monitor 10 may be configured to measure blood pressure by placement of a cuff 17 (FIG. 4) around a user's wrist, around a user's upper arm, or any other means of blood pressure measurement. The cuff 17 may measure the blood pressure of the user and convey an electronic signal to the monitor 10. Of course, other signals are contemplated within the spirit and scope of the invention. The general functioning of blood pressure monitor 10 is not described further herein as details thereof are well known in the art. While a particular housing shape and configuration of blood pressure monitor 10 is shown and described herein, it is understood any shape or configuration of blood pressure monitor 10 is fully contemplated according to the present invention.

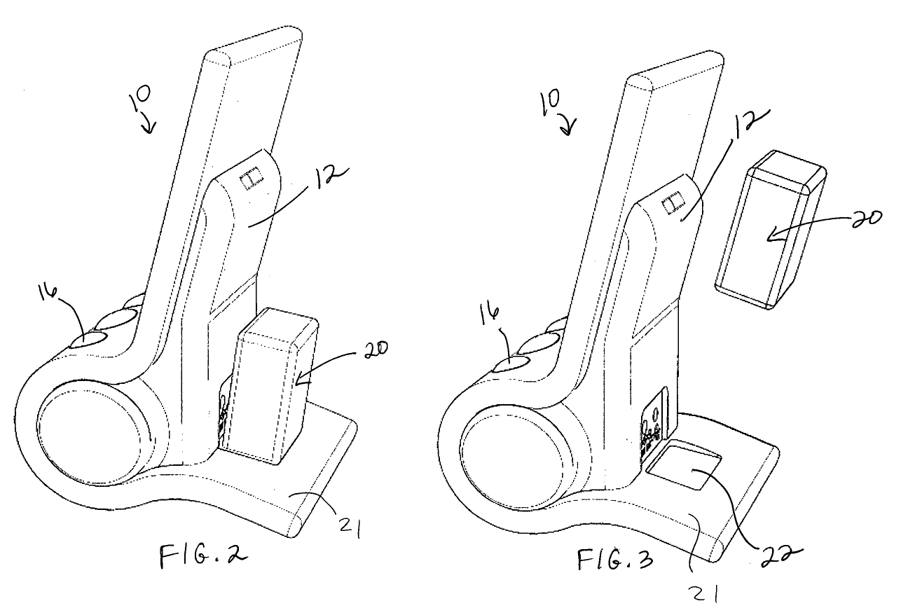

As shown in FIGS. 1-3, blood pressure monitor 10 of the present invention includes a removable power source 20, such as a battery, which may be mounted to housing 12. In FIG. 2, power source 20 is mounted on a rear portion of housing 12 opposite display 14 and control buttons 16. The power source 20 may be provided within an included angle defined by a base 21 of the housing 12 and the display 14 so that the battery 20 does not extend outside the overall dimensions of the blood pressure monitor 10 so that the battery 20 does not substantially increase the volume occupied by the monitor, thereby facilitating transportability of the monitor 10. Further, packaging for the monitor 10 may be minimized by avoiding an increase in the overall dimensions of the monitor 10. Additionally, by placement of the power source 20 between the base 21 and the display 14, the battery 20 does not detract from an overall appearance of the blood pressure monitor 10. Of course, other mounting locations of power source 20 on housing 12 could also be utilized.

According to one aspect of the present invention, power source 20 is sized to be received within a receptacle 22 provided in housing 12 (best shown in FIGS. 1 and 3). Power source 20 may be received and retained within receptacle 22 by a snap-fit arrangement, with an interference fit, a slide-in arrangement, using mating prongs and corresponding receiving areas (not shown), or by any other means of retaining power source 20 within receptacle 22 as long as desired by the user. Although a rectangular battery 20 and correspondingly-shaped receptacle 22 are shown herein, it is understood that battery 20 and receptacle 22 can have any shape or configuration in accordance with the present invention.

In one embodiment, receptacle 22 is configured to receive power source 20 such that power source 20 remains visible and accessible during engagement. In another embodiment, power source 20 could be received within a portion of housing 12, such as an internal compartment or the like, such that power source 20 may be partially to fully concealed when engaged. Power source 20 can be arranged to be manually removable by the user, such as simply by applying adequate force to disengage the snap-fit, interference fit, or other mating arrangement between power source 20 and receptacle 22. Alternatively, a push-button, sliding switch, or the like (not shown) and corresponding mechanism could be provided which, when actuated, ejects power source 20 from receptacle 22 for removal. Furthermore, a lock could be provided which retains power source 20 within receptacle 22 by any means to avoid unintended dislodgement.

According to the present invention, receptacle 22 may be provided in communication with control electronics 24 (FIG. 4) located within housing 12, such that connection of power source 20 to receptacle 22 provides power for the operation of blood pressure monitor 10. Blood pressure monitor 10 may also include a power cord 26 (FIG. 4) which could be connected thereto to provide power to blood pressure monitor 10 for use in one scenario (for example, but not limited to, home use), whereas power source 20 could be available to be connected to blood pressure monitor 10 for use in another scenario (for example, but not limited to, travel use). In this way, a user may be provided the versatility of using a power cord or removable power source 20, such as a battery, with their blood pressure monitor 10 whenever either option is most convenient, thus potentially increasing the portability, ease of use, and consequently the user compliance related to blood pressure monitor 10.

Receptacle 22 could also be provided in communication with an internal, rechargeable power source such as a rechargeable battery 28 (FIG. 4), such that connection of power source 20 to receptacle 22 for a period of time acts to replenish the internal power source. In this manner, power source 20 could be connected for a period of time, be subsequently removed, and then blood pressure monitor 10 would be able to operate without either power source 20 or a power cord connection.

The invention contemplates that the power source 20 may be utilized for powering various components within the housing 12. As discussed above, these components may include but are not limited to the display 14, the controller 24, the rechargeable battery 28, or the like. Additionally, the power source 20 may be utilized for powering a pump 30 within the housing 12 for pressurizing the cuff 17. For the embodiment illustrated, the power source 20 may impart power to the controller 24, which operates the pump 30 for pressurizing the cuff 17, which measures a blood pressure measurement of the user. The blood pressure measurement may be conveyed from the cuff 17 to the controller 24 and displayed upon the display 14, which is also powered by the power source 20.

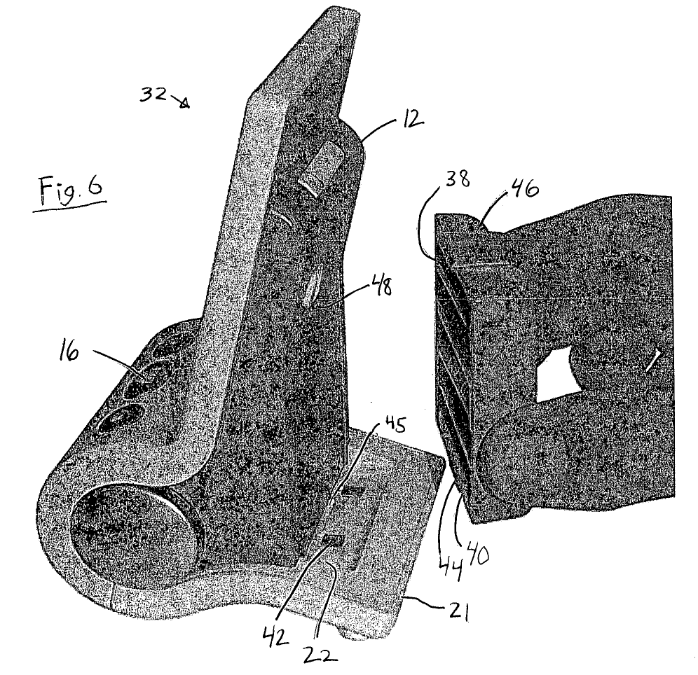

With reference now to FIGS. 5 and 6, another blood pressure monitor 32 is illustrated in accordance with the present invention. The blood pressure monitor 32 includes a battery pack 34, which includes a detachable housing portion 36 for receiving an array of batteries 38 within the housing portion 36. The housing portion 36 conceals the batteries 38 when assembled against a rear surface of the housing 12 and within the receptacle 22.

In one embodiment, the housing portion 36 is provided with a pair of electrical contacts 40, which are oriented to engage a corresponding pair of electrical contacts 42 within the receptacle 22 such that when the battery pack 34 is assembled to the housing 12, the array of batteries 38 are in electrical communication with the components of the housing 12 through the contacts 40, 42 of the housing portion 36 and the housing 12.

The housing portion 36 may include a tab 44, which is sized to be placed within a slot 45 in the receptacle 22. The housing portion 36 may also include a latch 46 with a leading edge sized to be received within a corresponding slot 48 within the backside of the housing 12. Thus, the housing portion 36 is assembled to the housing 12 by placing the tab 44 within the slot 45 and pivoting the leading edge of the latch 46 into the slot 48 thereby attaching the housing portion 36 to the housing 12. The latch 46 may be biased to an extended orientation by a spring or other suitable biasing member for securing the latch 46 to the slot 48 when assembled. The housing portion 36 may be disassembled from the housing 12 by depressing the latch 46 thereby disengaging the latch for removal of the battery pack 34.

While embodiments of the invention have been illustrated and described, it is not intended that these embodiments illustrate and describe all possible forms of the invention. Rather, the words used in the specification are words of description rather than limitation, and it is understood that various changes may be made without departing from the spirit and scope of the invention.

1. The blood pressure monitor of claim 12 further comprising:
a housing providing the base and the display;
a controller oriented within the housing; and
a blood pressure sensor external of the housing and in communication with the controller for providing the controller with a blood pressure measurement;
wherein the display is oriented on the housing in communication with the controller for receiving the blood pressure measurement from the controller, for displaying the blood pressure measurement, and the display extends generally upright from the base; and
wherein the removable power source is selectively attachable to the housing in communication with the controller and display for providing power to the controller and the display.

2. The blood pressure monitor of claim 12 wherein the power source further comprises a battery.

3. The blood pressure monitor of claim 12 wherein the power source further comprises an array of batteries.

4. The blood pressure monitor of claim 1 wherein the housing further comprises a receptacle for receiving the power source.

5. The blood pressure monitor of claim 1 wherein the removable power source attaches to the housing spaced apart from and rearward of the display.

6. The blood pressure monitor of claim 1 further comprising a rechargeable power source oriented within the housing that is recharged by the removable power source.

7. The blood pressure monitor of claim 1 wherein the power source further comprises:
a detachable housing portion; and
an array of batteries oriented within the housing portion.

8. The blood pressure monitor of claim 7 wherein the housing further comprises a first latch configuration and the housing portion further comprises a second latch configuration for cooperating with the first latch configuration for securing the housing portion to the housing.

9. The blood pressure monitor of claim 7 wherein the housing further comprises a pair of electrical nodes and the housing portion further comprises a corresponding pair of electrical nodes that are in electrical communication with the array of batteries and are aligned with the electrical nodes of the housing for conveying electrical power from the array of batteries to the housing.

10. The blood pressure monitor of claim 7 wherein the housing portion and the housing collectively conceal the array of batteries when the housing portion is attached to the housing.

11. The blood pressure monitor of claim 12 further comprising:
a housing having a first latch configuration and a pair of electrical nodes;
a controller oriented within the housing in electrical communication with the housing electrical nodes;
a blood pressure sensor external of the housing and in communication with the controller for providing the controller with a blood pressure measurement, wherein the display is oriented on the housing in communication with the controller for receiving the blood pressure measurement from the controller and for displaying the blood pressure measurement, the display being in electrical communication with the housing electrical nodes; and
a removable housing portion having a second latch configuration for cooperating with the first latch configuration for securing the housing portion to the housing, and a corresponding pair of electrical nodes that are aligned with the electrical nodes of the housing for electrical communication between the housing and housing portion nodes;
wherein the removable power furthre comprises at least one battery oriented in the housing portion in electrical communication with the pair of housing portion nodes for providing power to the controller and the display.

12. A blood pressure monitor comprising:
a base for supporting the blood pressure monitor upon an underlying support surface;
a display extending from the base for displaying a blood pressure measurement, the display and base forming an acute angle included between a rear surface of the display and the base; and
a removable power source attached to one of the base and the display in a region provided within the acute angle between the base and the display such that the removable power source does not extend outside the overall dimensions of the base and the display and does not obfuscate an overall appearance of the blood pressure monitor.

13. The blood pressure monitor of claim 1 wherein the removable power source is selectively attachable to the housing such that the removable power source is fixed from translation relative to the housing.

14. The blood pressure monitor of claim 4 wherein the receptacle is sized to receive and support the power source therein.

15. The blood pressure monitor of claim 5 wherein the removable power source attaches to the housing at an orientation that is adjacent to a rear surface of the housing.

16. The blood pressure monitor of claim 10 wherein the detachable housing portion partially conceals the array of batteries with an exposed region that is concealed only when attached to the housing.

17. The blood pressure monitor of claim 11 wherein one of the first and second latch configurations includes a leading edge and the other of the first and second latch configurations includes a slot for receiving the leading edge to prevent translation of the housing portion relative to the housing when the housing portion is attached to the housing.

18. The blood pressure monitor of claim 11 wherein the housing electrical nodes are in contact with the housing portion electrical contact nodes when the housing portion is attached to the housing, and the housing electrical nodes are not in contact with the housing portion contact nodes when the housing portion is detached from the housing.

19. The blood pressure monitor of claim 12 wherein the display is affixed to the base such that the acute angle included therebetween is not adjustable.

20. The blood pressure monitor of claim 12 wherein the base and display further comprise a unitary housing such that the acute angle is fixed for extending the display generally upright from the underlying support surface.

21. A blood pressure monitor comprising:
a base for supporting the blood pressure monitor upon an underlying support surface;
a display extending from the base for displaying a blood pressure measurement, the display and base forming an acute angle included between a rear surface of the display and the base; and
a removable battery attached to one of the base and the display such that the removable battery does not extend outside the overall dimensions of the base and the display and does not obfuscate an overall appearance of the blood pressure monitor.

22. A blood pressure monitor comprising:
a base for supporting the blood pressure monitor upon an underlying support surface;
a display extending from the base for displaying a blood pressure measurement, the display and base forming an acute angle included therebetween; and
a removable battery pack attached to one of the base and the display in a region provided within the acute angle between the base and the display such that the removable battery pack does not extend outside the overall dimensions of the base and the display and does not obfuscate an overall appearance of the blood pressure monitor;
wherein the display is affixed to the base such that the acute angle included therebetween is not movable.
